(19) United States
(12) Patent Application Publication
(10) Pub. No.: US 2026/0327290 A1
(43) Pub. Date: Oct. 22, 2026

(54) METHOD FOR RAPIDLY SEPARATING A SHEEP SERUM ALBUMIN BASED ON AN AQUEOUS TWO-PHASE SYSTEM

(71) Applicant: China Agricultural University, Beijing (CN)

(72) Inventors: Hao ZHANG, Beijing (CN); Xinfang KOU, Beijing (CN); Fazheng REN, Beijing (CN); Yunfei WANG, Beijing (CN); Lumeng CHAO, Beijing (CN); Jianfei WANG, Beijing (CN)

(73) Assignee: China Agricultural University, Beijing (CN)

(21) Appl. No.: 19/401,410

(22) Filed: Nov. 26, 2025

(30) Foreign Application Priority Data
Apr. 21, 2025 (CN) 202510496326.6

(51) Int. Cl. C07K 14/765

(52) U.S. Cl. CPC C07K 14/765

(57) ABSTRACT

A method for rapidly separating a sheep serum albumin based on an aqueous two-phase system is provided. The aqueous two-phase system consists of ethanol, sodium dihydrogen phosphate, and water, or the aqueous two-phase system consists of ethanol, sodium dihydrogen phosphate, water, and sodium chloride. By adjusting system components and operating conditions, the separation efficiency is significantly improved, the efficient extraction and application of the sheep serum albumin are achieved, and a new path for high-value utilization technologies of animal blood resources is developed.

### CROSS REFERENCE TO THE RELATED APPLICATIONS

This application is based upon and claims priority to Chinese Patent Application No. 202510496326.6, filed on Apr. 21, 2025, the entire contents of which are incorporated herein by reference.

### TECHNICAL FIELD

The present disclosure belongs to the technical field of biological engineering technology, and specifically to a method for rapidly separating a sheep serum albumin based on an aqueous two-phase system.

### BACKGROUND

As a crucial by-product in livestock slaughtering, sheep blood is rich in nutrients and bioactive components including proteins, amino acids, enzymes, vitamins, hormones, minerals, carbohydrates, and lipids. However, the utilization efficiency of sheep blood remains alarmingly low. Most slaughterhouses directly discharge the blood, resulting in massive waste of valuable protein resources and environmental pollution.

Sheep blood is rich in proteins, amino acids, vitamins, and various bioactive substances, herein the proteins are its main component, and the content reaches 17%-19%. Albumins are one of the most important proteins in the sheep blood, and account for 60% of the total plasma proteins (Cheng Chi and Cai Yongfeng. Development and Utilization of Edible Animal Blood Resources [J]. Food and Fermentation Industry, 1998, 24(3): 7.). The sheep serum albumin contains abundant essential amino acids and has the higher bioavailability, which makes it of great value in the development of nutritional supplements and healthy foods. Due to its excellent nutritional ingredients, the sheep serum albumin has broad application prospects in postoperative repair, it may quickly supplement the proteins, promote patients' recovery processes, and help accelerate tissue repair and recovery.

At present, separation methods of the sheep serum albumin mainly include a salt fractionation method, an organic solvent precipitation method, an ultrafiltration method, and a chromatographic separation method and the like. However, these traditional methods have certain limitations in practical applications. For example, the salt fractionation method is widely used due to its simple operations, but it requires the use of high-concentration salt solution to achieve protein precipitation, this process may damage the purity of the proteins and desalination working procedures are often complex and time-consuming. Chinese patent CN201710059284.5 uses a saturated ammonium sulfate fractional precipitation method to obtain a bovine serum albumin, but the preparation process is cumbersome and requires chromatographic column desalination, and the cost is improved. The ultrafiltration method uses a selective permeable membrane to remove small-molecular impurities, but it is easy to cause membrane pollution and blockage, thereby the recovery efficiency and separation efficiency are reduced. The organic solvent precipitation method is the most commonly used technique for separating plasma albumins at present, and the separation is based on protein isoelectric point precipitation, but the treating process may cause denaturation of the sheep serum albumin, and its functional characteristics are affected. For example, Chinese patent CN200410077667.8 uses a cold ethanol method to separate albumins from human plasma protein waste components, but this method is limited by strict separation conditions and does not specify the purity and structural characteristics of the albumins obtained. The chromatographic separation technology shows certain advantages in the field of protein extraction, but the high device investment limits its possibility for large-scale applications. Chinese patent CN202410514938.9 discloses a method for separating a canine albumin from canine blood using two times of chromatography. Although the purity of the proteins is improved, the cost of chromatography media is relatively high, which limits widespread applications of this method.

Therefore, how to improve the separation purity and reduce the cost under the premise of maintaining the activity of the sheep serum albumin still remains a key challenge in current researches and applications.

### SUMMARY

In order to overcome deficiencies of existing technologies, improve the separation efficiency of the sheep serum albumin and reduce the cost, an efficient and low-cost sheep serum albumin separation technology is developed. The present invention provides a method for rapidly separating a sheep serum albumin based on an ethanol-salt aqueous two-phase system. By adjusting system components and operating conditions, the separation efficiency is significantly improved, the efficient extraction and application of the sheep serum albumin are achieved, and a new path for high-value utilization technologies of animal blood resources is developed.

To achieve the above purpose, the technical scheme of the present invention is realized as follows:

A first aspect of the present disclosure provides a method for rapidly separating a sheep serum albumin based on an aqueous two-phase system, characterized by the following steps:
(1) adding the aqueous two-phase system to sheep plasma, stilly placing after stirring a mixture obtained, centrifuging, and collecting upper phase solution, wherein the aqueous two-phase system consists of ethanol, sodium dihydrogen phosphate, and water, or the aqueous two-phase system consists of ethanol, sodium dihydrogen phosphate, water, and sodium chloride;
(2) removing the ethanol from the upper phase solution;

Further, the molecular weight of the sheep serum albumin is 66-68 kDa, preferably 66 kDa.

Further, an acquisition method for sheep plasma is as follows; an anticoagulant is added to sheep blood, it is centrifuged, and a supernatant is retained, to obtain the sheep plasma.

Further, the sheep blood needs to undergo inspection and quarantine before use, and may only be used as a raw material for extracting the sheep serum albumin after passing the inspection.

Further, the anticoagulant is selected from ethylenediamine tetraacetic acid, sodium citrate, and heparin, and further preferably it is 3.8% sodium citrate.

Further, the volume ratio of the anticoagulant to the sheep blood is 1:(5-20). For example, 1:5, 1:7, 1:9, 1:10, 1:11, 1:12, 1:13, 1:14, 1:16, 1:18 or 1:20, preferably 1:10.

Further, the sheep plasma extracted may be freeze-stored at −20° C., and when it is ready for use, the sheep plasma frozen may be melted in a water bath at 37° C. before the use.

Preferably, the mass percentage of the sheep plasma in the mixture is 1-5%. For example, 1%, 2%, 3%, 4% or 5%, more preferably 2~4%.

Preferably, the mass percentage of the ethanol in the mixture is 25-35%. For example, 25%, 26%, 27%, 28%, 29%, 30%, 32%, 33% or 35%, more preferably 25~30%.

Preferably, the mass percentage of the sodium dihydrogen phosphate in the mixture is 16-24%. For example, 16%, 17%, 18%, 19%, 20%, 22% or 24%, more preferably 16~20%.

Preferably, the mass percentage of the sodium chloride in the mixture is 0~10%. For example, 0%, 1%, 2%, 3%, 4%, 5%, 6%, 7% or 10%, more preferably 1-5%.

In a specific implementation mode of the present invention, in the aqueous two-phase system, the mass percentage of the sheep plasma in the mixture is 3%, the mass percentage of the ethanol in the mixture is 30%, the mass percentage of the sodium dihydrogen phosphate in the mixture is 20%.

In a specific implementation mode of the present invention, in the aqueous two-phase system, the mass percentage of the sheep plasma in the mixture is 4%, the mass percentage of the ethanol in the mixture is 28%, the mass percentage of the sodium dihydrogen phosphate in the mixture is 18%, and the mass percentage of the sodium chloride in the mixture is 3%.

Preferably, the standing time is 10-60 min. For example, 10 min, 15 min, 20 min, 30 min, 35 min, 40 min, 45 min, 50 min or 60 min.

Preferably, the centrifugation conditions are: the centrifugation temperature is 1-10° C. (For example, 1° C., 2° C., 3° C., 4° C., 5° C., 6° C., 7° C., 8° C., 9° C. or 10° C.), the centrifugation speed is 2500-4500 rpm (For example, 2500 rpm, 3000 rpm, 3500 rpm, 4000 rpm or 4500 rpm), and the centrifugation time is 5-25 min (For example, 5 min, 10 min, 15 min, 20 min or 25 min).

In a specific implementation mode of the present invention, the centrifugation conditions are: the centrifugation temperature is 4° C., the centrifugation speed is 3500 rpm, and the centrifugation time is 15 min.

Further, the step (1) is carried out at room temperature, the room temperature is 20-35° C.

Further, the method for removing the ethanol in Step (2) is a dialysis technology, and a dialysis bag with a cut-off molecular weight of 10 kDa is used in the dialysis process.

Further, step (2) further includes a freeze-drying operation.

Preferably, the conditions for the freeze-drying operation are: the freeze-drying temperature is −90-70° C. (For example, −90° C., −85° C., −80° C., −75° C. or −70° C.), and the freeze-drying time is 36-60 h (For example, 36 h, 40 h, 44 h, 46 h, 48 h, 50 h, 52 h, 56 h or 60 h).

In a specific implementation mode of the present invention, the method includes the following steps.
(1) After inspection and quarantine of fresh sheep blood is passed, an anticoagulant which is 10% of the original blood volume is added, and centrifuged at 4° C. and 3500 rpm for 15 min, then a supernatant is taken out, and freeze-stored at −20° C.;
(2) when it is ready for use, sheep plasma frozen is taken out and melted in a water bath at 37° C., to obtain sheep plasma;
(3) ethanol, sodium dihydrogen phosphate, and water are successively added to the sheep plasma to form an aqueous two-phase system, the mass percentage of the sheep plasma in the mixture is 3%, the mass percentage of the ethanol in the mixture is 30%, and the mass percentage of the sodium dihydrogen phosphate in the mixture is 20%, the remaining part of the aqueous two-phase system is composed of the water, and it is stilly placed after being stirred;
(4) after being stilly placed, two phases are separated, and upper phase solution is collected; and
(5) the ethanol is removed from the upper phase solution, and freeze-dried, to obtain the sheep serum albumin.

In another aspect of the present invention, an application of the sheep serum albumin prepared by the above method in a food or a biochemical material is provided.

Preferably, the food is a nutritional supplement or a food additive.

Hydrolysates of the sheep serum albumin are rich in various amino acids, peptides, and trace elements, it is suitable for improving the nutritional value of the food as the nutritional supplement; at the same time, it has good gel, emulsification, moisturizing and other functional characteristics, and it may be used as the food additive to improve the taste and quality of the food.

Preferably, the biomaterial may be used as a raw material for protein electrophoresis or cell culture, an antigen for immune detection, a biofilm, biological glue, or a drug carrier.

In the protein electrophoresis, it may serve as an important experimental material that may provide a reliable reference standard for separation and analysis of other proteins under the action of an electric field, and help researchers accurately understand the characteristics and differences of the proteins. In the process of the cell culture, using the sheep serum albumin as an additive of a cell culture medium may provide necessary nutrient substances and growth factors for cell growth, and create an ideal microenvironment for the cell culture.

In terms of immune detection, the sheep serum albumin may serve as an immunogen for preparing antibodies, and these antibodies may accurately recognize and bind to the sheep serum albumin or other proteins related to its structure, it plays a crucial role in disease diagnosis, quality test of biological products, and other fields.

In the preparation of the biomaterial, the sheep serum albumin may be used to manufacture products such as the biological glue and the biofilm, and it plays a role in tissue engineering, wound healing and other fields.

In the field of the drug carrier, the sheep serum albumin may serve as the drug carrier, it accurately delivers drugs to specific tissues or cells, thereby the drug efficacy is improved and side effects are reduced.

### The Present Disclosure has the Following Beneficial Effects:

(1) The present invention develops a sheep serum albumin extraction method by experimental screening, and it has the outstanding characteristics of short process, simple operation, and high production efficiency. Especially, by using the aqueous two-phase system with specific components and ratios, the protein aggregation or denaturation may be effectively prevented in the extraction process, the quality of the sheep serum albumin is guaranteed greatly, the recovery rate and purity of albumin products may also be improved significantly, the utilization rate of sheep blood is increased greatly, and the environmental pollution caused by the direct discharge of the sheep blood is avoided. By means of these advantages, this method and the sheep serum albumin extracted by this method are widely used in many fields such as foods and biomedicines.
(2) The present invention uses mild conditions for protein extraction, and the entire operation process is rigorous and controllable, and the damage to the protein structure is avoided, so that the protein always maintains its original excellent activity.
(3) The present invention compares the albumin functions of different species, and provides a theoretical basis for future applications.

### BRIEF DESCRIPTION OF THE DRAWINGS

The embodiments of the present invention are described in detail below with reference to the accompanying drawings:

FIG. 1 is an aqueous two-phase phase diagram of different aqueous two-phase systems.

FIG. 2 is a result diagram of phase separation processes of the different aqueous two-phase systems.

FIGS. 3A-3D are electrophoresis diagrams of each layer system in the phase separation processes of the different aqueous two-phase systems, FIG. 3A is an electrophoresis diagram of plasma protein separation in an ethanol and sodium dihydrogen phosphate system; FIG. 3B is an electrophoresis diagram of plasma protein separation in an ethanol and dipotassium hydrogen phosphate system; FIG. 3C is an electrophoresis diagram of plasma protein separation in an ethanol and sodium citrate system; FIG. 3D is an electrophoresis diagram of plasma protein separation in an ethanol and sodium carbonate system; M is a protein molecular weight standard; and the plasma is sheep plasma extracted.

FIGS. 4A-4B are extraction rate and purity analysis diagrams of a sheep serum albumin separated by the different aqueous two-phase systems, FIG. 4A is a recovery rate diagram of the sheep serum albumin separated by the aqueous two-phase systems with different types of salts, and FIG. 4B is a purity diagram of the sheep serum albumin separated by the aqueous two-phase systems with the different types of salts.

FIG. 5 is a result diagram of sheep serum albumin redissolved after extraction treatment by the different aqueous two-phase systems and freeze-drying.

FIG. 6 is a sodium dodecyl sulfate polyacrylamide gel electrophoresis (SDS-PAGE) diagram of the sheep serum albumin separated by the ethanol-sodium dihydrogen phosphate aqueous two-phase system, lane 1: M is the protein molecular weight standard; lane 2: the plasma is the sheep plasma; lane 3: Sample A; and lane 4: the standard sample is a sheep serum albumin standard sample.

FIGS. 7A-7B are infrared spectrum and fluorescence spectrum diagrams of the sheep serum albumin separated by the ethanol-sodium dihydrogen phosphate aqueous two-phase system; FIG. 7A is an infrared spectrum; FIG. 7B is a fluorescence spectrum diagram.

FIG. 8 is a differential scanning calorimetry (DSC) of the sheep serum albumin separated by the ethanol-sodium dihydrogen phosphate aqueous two-phase system.

FIG. 9 is a comparison diagram of emulsion stability of different species of albumins.

FIG. 10 is a comparison diagram of gel strength of the different species of albumins.

Note: a, b, and c in the drawings all represent p<0.05.

### DETAILED DESCRIPTION OF THE EMBODIMENTS

The following provides a clear and comprehensive description of the technical solutions in the embodiments of the present invention, using the accompanying drawings. It should be noted that the described embodiments represent only some of the embodiments of the present invention, not all. Based on these embodiments, any other embodiments obtained by those skilled in the art without making inventive contributions fall within the scope of protection of the present invention.

### Embodiment 1: Screening of Combination of Aqueous Two-Phase System

### 1. Screening Method for Aqueous Two-Phase System:

(1) firstly, a certain amount of an inorganic salt (m₁) (the inorganic salt was potassium bicarbonate, sodium carbonate, sodium sulfate, sodium citrate, sodium acetate, dipotassium hydrogen phosphate or sodium dihydrogen phosphate respectively) was weighed and a certain amount of distilled water (m₂) was added so that it was just dissolved;
(2) ethanol (m₃) was dropwise added until mixed solution just became turbid, and it was stilly placed for equilibrium;
3) the equilibrium process was maintained in a state in which adding a drop of water immediately made the solution clear, and adding a little more ethanol immediately made the solution turbid again;
4) the above operation was repeated, it was observed whether phase separation was possible, and the mass fraction of each component in the solution at a turbidity point was calculated according to the following formula.

$$Inorganic salt mass fraction ( \% ) = \frac{{m}_{1}}{{m}_{1} + {m}_{2} + {m}_{3}} \times 100$$

$$Ethanol mass fraction ( \% ) = \frac{{m}_{3}}{{m}_{1} + {m}_{2} + {m}_{3}} \times 100$$

The results were shown in Table 1.

TABLE 1
Phase formation conditions of different types of salts

| | Types of salts | Phase formation conditions |
|---|---|---|
| Ethanol | Potassium bicarbonate | A salt precipitation phenomenon |
| | | occurs and a phase is not formed |
| | Sodium carbonate | Stable phase formation |
| | Sodium sulfate | A salt precipitation phenomenon |
| | | occurs and a phase is not formed |
| | Sodium citrate | Stable phase formation |
| | Sodium acetate | A salt precipitation phenomenon |
| | | occurs and a phase is not formed |
| | Dipotassium hydrogen | Stable phase formation |
| | phosphate | |
| | Sodium dihydrogen | Stable phase formation |
| | phosphate | |

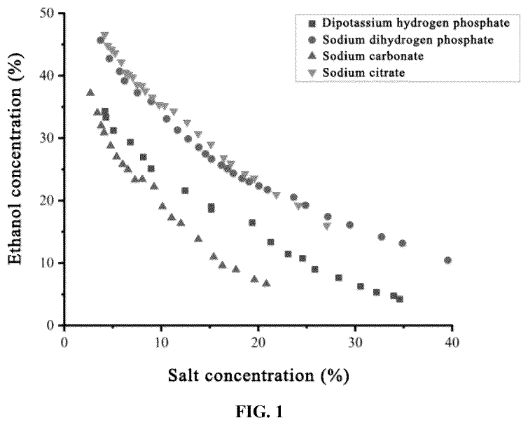

The mass fraction of the salt (dipotassium hydrogen phosphate, sodium dihydrogen phosphate, sodium citrate, sodium carbonate) that was stable for phase formation with the ethanol was calculated according to the method in Step 4) above, and an aqueous two-phase phase diagram (as shown in FIG. 1) was drawn to determine the phase separation conditions.

### Embodiment 2: Screening and Separation of Aqueous Two-Phase System Combination of Sheep Serum Albumin

### 1. Extraction Method for Sheep Serum Albumin

(1) after inspection and quarantine of fresh sheep blood was passed, an anticoagulant which was 10% of the original blood volume was added, and centrifuged at 4° C. and 3500 rpm for 15 min, then a supernatant was taken out, and freeze-stored at −20° C.;
(2) when it was ready for use, sheep plasma frozen was taken out and melted in a water bath at 37° C., to obtain sheep plasma;
(3) ethanol, sodium dihydrogen phosphate, and water were successively added to the sheep plasma to form an aqueous two-phase system, the mass percentage of the sheep plasma in the mixture was 3%, the mass percentage of the ethanol in the mixture was 30%, and the mass percentage of the sodium dihydrogen phosphate in the mixture was 20%, the remaining part of the aqueous two-phase system was composed of the water, and it was stilly placed after being stirred;
(4) after being stilly placed, two phases were separated, and upper phase solution was collected; and
(5) the ethanol was removed from the upper phase solution, and freeze-dried, to obtain the sheep serum albumin.

### 2. Determination Method for Content of Sheep Serum Albumin

The total protein content of the ethanol solution collected in Step (4) was determined by using a coomassie brilliant blue method and the albumin content was determined by using a high performance liquid chromatography (HPLC).

The HPLC conditions were as follows: it was a C8 column (4.6×250 mm, 5 μm), the column temperature was 40° C., a mobile phase A was 0.01% trifluoroacetic acid (TFA) aqueous solution, a mobile phase B was 0.01% TFA acetonitrile solution, it was gradient-eluted, the flow rate was 0.8 mL/min, the sample injection volume was 20 μl, and the determination wavelength was 276 nm.

The recovery rate and purity of the sheep serum albumin were calculated by using the following method:

$$Recovery rate ( \% ) = \frac{Albumin content in sample}{Albumin content in plasma added} \times 100$$

$$Purity ( \% ) = \frac{Albumin content in sample}{Total protein content in sample} \times 100$$

### 3. Result

### (1) Screening of Inorganic Salt Types

The types of inorganic salts in the aqueous two-phase system were screened, and the sodium dihydrogen phosphate in Step (3) of the extraction method was replaced with dipotassium hydrogen phosphate, sodium citrate, or sodium carbonate, and the other extraction steps were the same as the above extraction method for the sheep serum albumin.

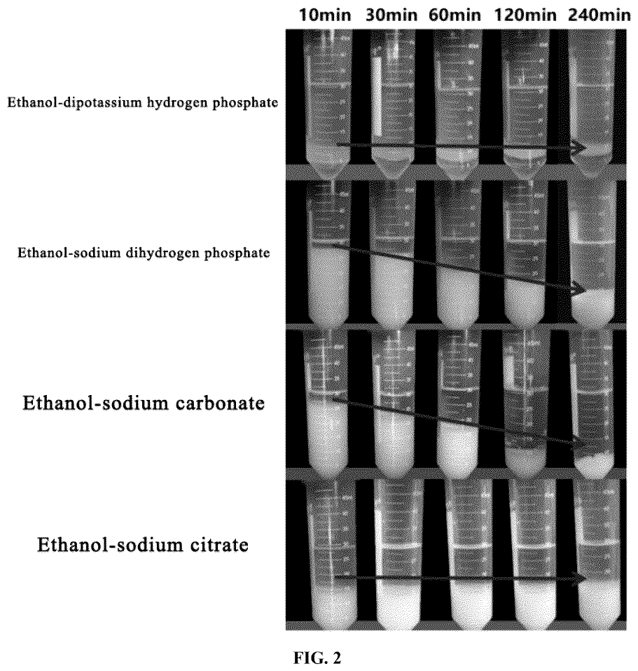

FIG. 2 showed a result of the phase separation process of the ethanol in the aqueous two-phase system with the dipotassium hydrogen phosphate, sodium dihydrogen phosphate, sodium citrate, and sodium carbonate. Herein, the phase separation of the sheep plasma in the ethanol and sodium dihydrogen phosphate system was complete already within 30 min and remained relatively stable, only a small amount of interfacial precipitation was generated; although the phase separation speed of the sheep plasma was fast in the ethanol and sodium carbonate system, a large amount of proteins was denaturated and a precipitate was generated; the phase separation of the sheep plasma in the ethanol and dipotassium hydrogen phosphate system and the ethanol and sodium citrate system took 240 min to complete. At the same time, after the phase separation of the ethanol and dipotassium hydrogen phosphate system was complete, it was accompanied by a large amount of protein precipitates, while a salt precipitation phenomenon occurred after the phase separation of the ethanol and sodium citrate system was complete.

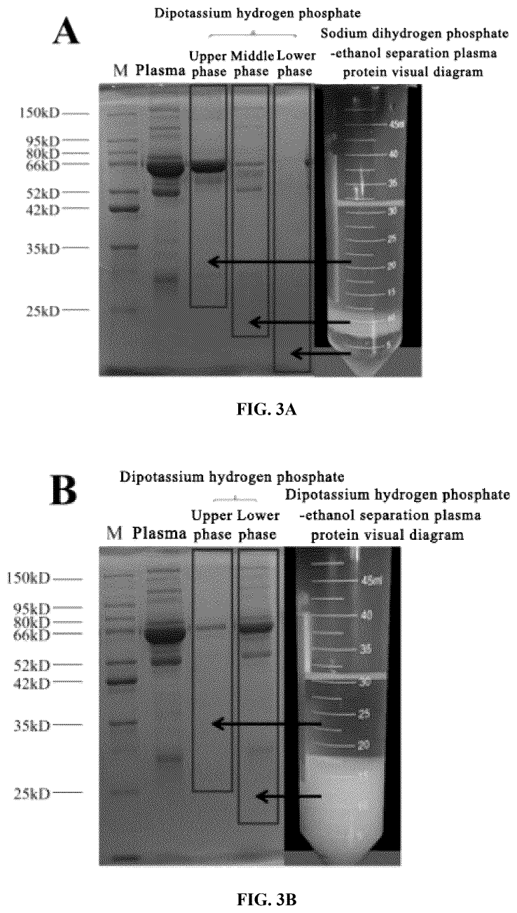

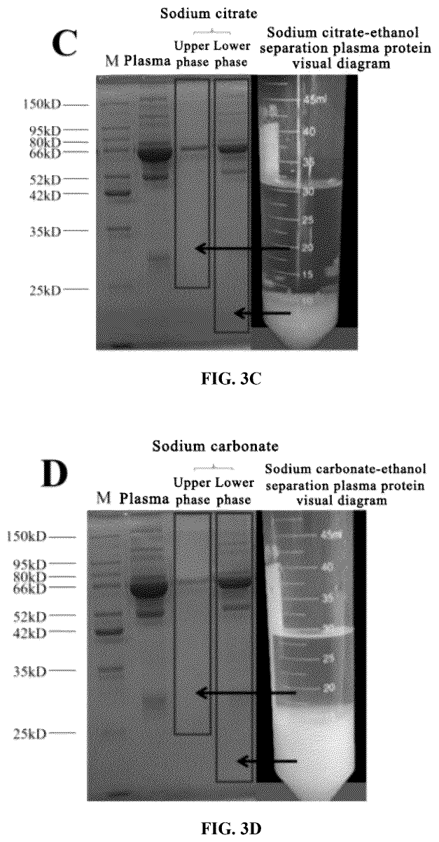

Electrophoresis detection was performed for each layer system in the process of the phase separation of the different systems, and sodium dodecyl sulfonate-polyacrylamide gel electrophoresis was used, the specific test results were shown in FIGS. 3A-3D. In the system corresponding to FIG. 3A, almost all proteins with a molecular weight of 66 kD were extracted from the upper phase, and only a small amount of proteins were present in the middle phase. In the systems involved in FIGS. 3B-3D, the proteins with the molecular weight of 66 kD underwent the denaturation during the phase separation in other aqueous two-phase systems and precipitated in the lower phase, only a small amount of proteins were extracted from the upper phase.

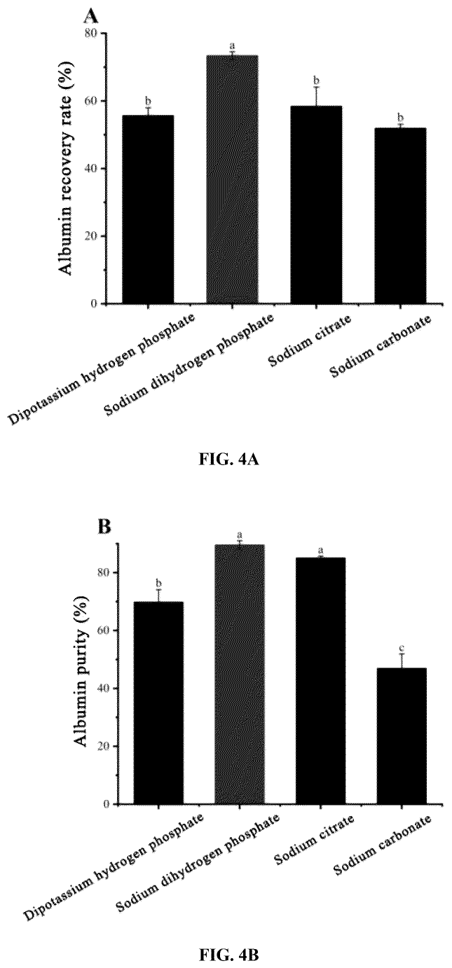

The result of the recovery rate and purity of the sheep serum albumin in the aqueous two-phase system composed of the ethanol, dipotassium hydrogen phosphate, sodium dihydrogen phosphate, sodium citrate, and sodium carbonate were shown in FIGS. 4A-4B. Herein the aqueous two-phase system composed of the ethanol and sodium dihydrogen phosphate showed the best separation effect on the sheep serum albumin, the recovery rate might reach 73.36%, and the purity might reach 89.52%.

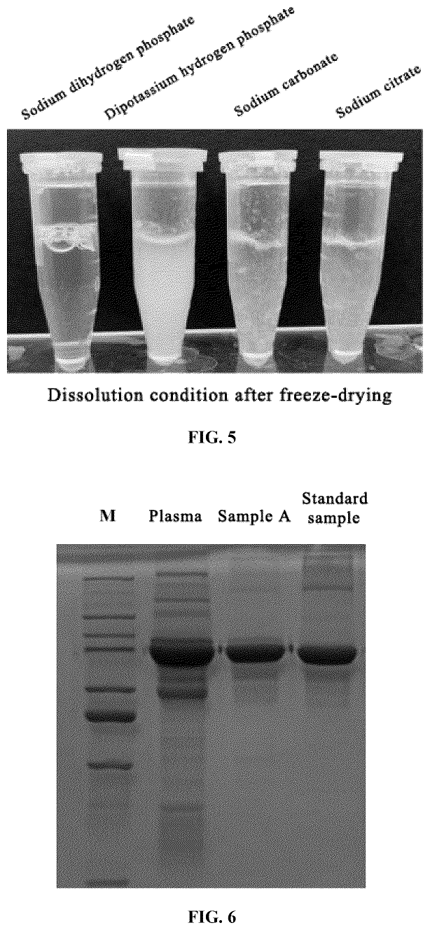

The sheep serum albumin treated with the different extraction systems and freeze-dried was redissolved to prepare aqueous solution with a concentration of 10 mg/mL, the results were shown in FIG. 5. The sheep serum albumin extracted in the ethanol-sodium dihydrogen phosphate extraction system had the most ideal dissolution effect, and the solution obtained was clear and transparent. In contrast, when the ethanol-sodium citrate and ethanol-sodium carbonate extraction systems were used, the solution was more turbid and contained obvious insoluble substances. However, when the ethanol-dipotassium hydrogen phosphate extraction system was used, the solution became even more turbid.

Therefore, from the above results, it might be seen that the ethanol-sodium dihydrogen phosphate system had the best effect on the separation and extraction of the sheep serum albumin from the sheep plasma. Subsequently, this system was selected and its optimal component ratio was further explored, as to determine the optimal process conditions.

### (2) Screening of Sheep Plasma Ratio

The ratio of the sheep plasma added to the aqueous two-phase system was screened, and the mass percentage of the sheep plasma in Step (3) of the extraction method was replaced with 1%, 2%, 3%, 4% and 5% of the mixture respectively. Only the ratio of the sheep plasma was changed, and the other extraction steps were the same as the above extraction method.

The screening result of the sheep plasma ratio was shown in Table 2. When the mass percentage of the ethanol and sodium dihydrogen phosphate in the mixture was constant, the recovery rate of the sheep serum albumin was highest while the mass percentage of the sheep plasma added to the mixture was 4%.

TABLE 2
Result analysis of recovery rate of sheep serum albumin
by different ratios of sheep plasma

| Sheep plasma | Ethanol | Sodium dihydrogen | |
|---|---|---|---|
| mass | mass | phosphate | Recovery |
| fraction (%) | fraction (%) | mass fraction (%) | rate (%) |
| 1 | 30 | 20 | 59.22 ± 0.72 |
| 2 | 30 | 20 | 61.80 ± 0.69 |
| 3 | 30 | 20 | 64.28 ± 1.16 |
| 4 | 30 | 20 | 69.25 ± 0.44 |
| 5 | 30 | 20 | 57.28 ± 0.94 |

### 3) Screening of Ethanol Ratio

The ratio of the ethanol added to the aqueous two-phase system was screened, the mass percentage of the ethanol in Step (3) of the extraction method was replaced with 25%, 28%, 30%, 32% and 35% of the mixture respectively, the mass percentage of the sheep plasma in the mixture was set to 4%, and the other extraction steps were the same as the above extraction method.

The screening result of the ethanol ratio was shown in Table 3. When the mass percentage of the sheep plasma and sodium dihydrogen phosphate in the mixture was constant, the recovery rate of the sheep serum albumin was highest while the mass percentage of the ethanol added to the mixture was 28%.

TABLE 3
Result analysis of recovery rate of sheep serum albumin
by different ratios of ethanol

| Ethanol | Sheep plasma | Sodium dihydrogen | |
|---|---|---|---|
| mass | mass | phosphate | Recovery |
| fraction (%) | fraction (%) | mass fraction (%) | rate (%) |
| 25 | 4 | 20 | 62.67 ± 0.78 |
| 28 | 4 | 20 | 72.39 ± 1.09 |
| 30 | 4 | 20 | 63.71 ± 0.52 |
| 32 | 4 | 20 | 34.89 ± 2.28 |
| 35 | 4 | 20 | 14.74 ± 0.46 |

### 4) Screening of Sodium Dihydrogen Phosphate Ratio

The ratio of the sodium dihydrogen phosphate added to the aqueous two-phase system was screened, the mass percentage of the sodium dihydrogen phosphate in Step (3) of the extraction method was replaced with 16%, 18%, 20%, 22% and 24% of the mixture respectively, the mass percentage of the sheep plasma in the mixture was set to 4%, the mass percentage of the ethanol in the mixture was set to 28%, and the other extraction steps were the same as the above extraction method.

The screening result of the sodium dihydrogen phosphate ratio was shown in Table 4. When the mass percentage of the sheep plasma and ethanol in the mixture was constant, the recovery rate of the sheep serum albumin was highest while the mass percentage of the sodium dihydrogen phosphate added to the mixture was 18%.

TABLE 4
Result analysis of recovery rate of sheep serum albumin
by different ratios of sodium dihydrogen phosphate

| Sodium dihydrogen | Sheep plasma | Ethanol | |
|---|---|---|---|
| phosphate | mass | mass | Recovery |
| mass fraction (%) | fraction (%) | fraction (%) | rate (%) |
| 16 | 4 | 28 | 76.47 ± 2.29 |
| 18 | 4 | 28 | 82.16 ± 3.43 |
| 20 | 4 | 28 | 69.99 ± 0.34 |
| 22 | 4 | 28 | 57.97 ± 1.70 |
| 24 | 4 | 28 | 43.26 ± 1.35 |

### 5) Addition of Multiple Inorganic Salts

The quantity and quality of inorganic salts added to the aqueous two-phase system were screened. In Step (3) of the extraction method, the mass percentage of the sheep plasma in the mixture was set to 4%, the mass percentage of the ethanol in the mixture was set to 28%, and the mass percentage of the sodium dihydrogen phosphate in the mixture was set to 18%. The different ratios of sodium chloride were added to the aqueous two-phase system, and the mass percentage of the sodium chloride in the mixture was 0%, 3%, 5%, 7% and 10%, and the other extraction steps were the same as the above extraction method.

The screening result of the sodium chloride ratio was shown in Table 5. In the aqueous two-phase system, when the mass percentage of the sheep plasma in the mixture was 4%, the mass percentage of the ethanol in the mixture was 28%, the mass percentage of the sodium dihydrogen phosphate in the mixture was 18%, and the mass percentage of the sodium chloride in the mixture was 3%, the aqueous two-phase system formed at this moment had the best separation effect on the sheep serum albumin, and the recovery rate might reach 92.05%.

TABLE 5
Result analysis of recovery rate of sheep serum albumin
by different ratios of sodium chloride

| Sodium | Sheep | Ethanol | Sodium | |
|---|---|---|---|---|
| chloride | plasma | mass | dihydrogen | Recovery |
| mass | mass | fraction | phosphate mass | rate |
| fraction (%) | fraction (%) | (%) | fraction (%) | (%) |
| 0 | 4 | 28 | 18 | 83.34 ± 1.09 |
| 3 | 4 | 28 | 18 | 92.05 ± 2.80 |
| 5 | 4 | 28 | 18 | 80.61 ± 0.92 |
| 7 | 4 | 28 | 18 | 55.57 ± 3.44 |
| 10 | 4 | 28 | 18 | 50.21 ± 0.73 |

### Embodiment 3: Structural Identification and Quality Analysis of Sheep Serum Albumin

Sodium dodecyl sulfonate-polyacrylamide gel electrophoresis was used, to perform comparison detection of protein molecular weight standards on standard samples of sheep plasma and sheep serum albumin (Solarbio, SP084) and a sample A prepared according to the extraction conditions in Embodiment 2 (the mass percentage of the sheep plasma in the mixture was 4%, the mass percentage of the ethanol in the mixture was 28%, the mass percentage of the sodium dihydrogen phosphate in the mixture was 18%, and the mass percentage of the sodium chloride in the mixture was 3%).

As shown in FIG. 6, electrophoresis results showed that the molecular weight of the albumin obtained from the sample A was consistent with a standard albumin, it was approximately 66 kDa, and the electrophoretic purity was higher than 90%.

Fourier transform infrared absorption spectroscopy was used to analyze the albumin of the sample A and the sheep serum albumin standard sample by a Fourier infrared spectrometer, and the spectrum of the sample was scanned in a frequency scanning range of 400-4000 cm⁻¹ with a resolution ratio of 4 cm⁻¹.

A fluorescence spectrometer was used to draw fluorescence spectra of the albumin of the sample A and the sheep serum albumin standard sample, the excitation wavelength was 280 nm, the emission wavelength was 300-450 nm, and the excitation and emission slits were both 3 nm.

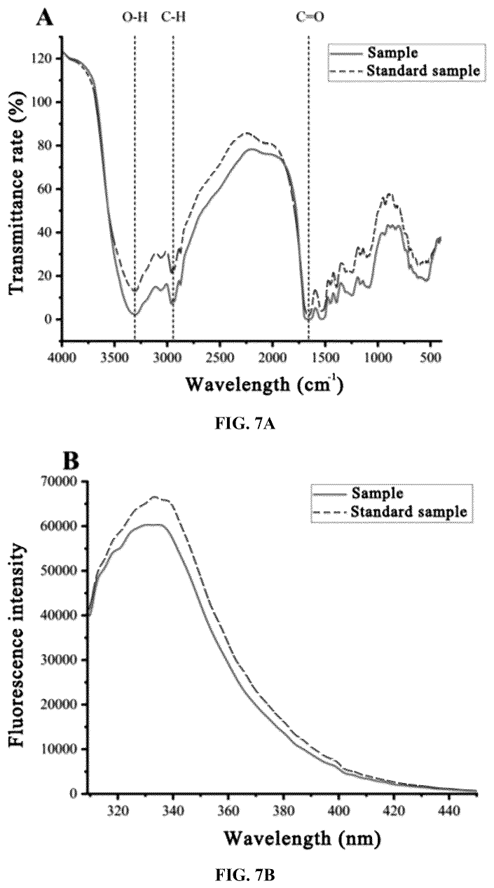

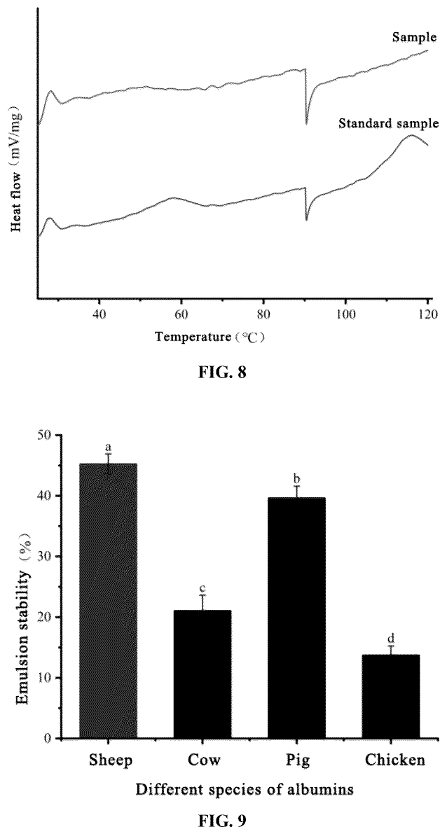

The results were shown in FIGS. 7A-7B, the albumin structure of the sample A was consistent with the standard sample, it was indicated that the extraction method of Embodiment 2 caused less damage to the protein structure.

### Embodiment 4: Stability Determination

The thermodynamic stability of the sheep serum albumin in the sample A was determined by a differential scanning calorimetry and compared with a standard albumin.

Approximately 8 mg of albumin powder and the sample A were weighed separately, and placed in an aluminum crucible for tabletting. A DSC instrument was placed, the nitrogen flow rate was 50 ml/min, a temperature program was set to 20-120° C., and the heating rate was 10° C./min.

The results were shown in FIG. 8, albumin molecules of the sample A had the same thermodynamic stability as the standard sample, its secondary structure was intact and the heat resistance was good, the stability requirements of biological products were met. This result further verified the protective effect of this extraction method on the native conformation of proteins.

### Embodiment 5: Protein Emulsibility and Emulsion Stability

The sample A and other different species of albumin pure samples were dissolved in deionized water to prepare 1 mg/mL of solution, and the other types of the albumin pure samples included bovine, porcine, and chicken albumins, the samples were emulsified by using a high-speed shearing machine at a rotation speed of 10000 r/min, corn oil was slowly added to the solution while homogenized, and finally the ratio of a water phase and an oil phase was 3:1. After the homogenization was completed, 50 μl of liquid was taken from the bottom of a tube and added to 5 mL of SDS solution (0.1%, w/v) at 0 min and 10 min respectively. Then the absorbance of the solution diluted was determined at 500 nm.

The calculation formula for emulsion stability (ESI) was as follows:

$$ESI ( \% ) = \frac{{A}_{10}}{{A}_{0}} \times 100$$

Herein, A₀—absorbance value at 0 min; A₁₀—absorbance value at 10 min.

The results were shown in FIG. 9, the sheep serum albumin had the significantly better emulsion stability than the bovine, porcine, and chicken albumins, this indicated that the sheep serum albumin had the better emulsifying properties and might provide the higher performance in food processing and related applications.

### Embodiment 6: Determination of Protein Gel Strength

### 1. Preparation of Albumin Gel

(1) The sample A was dissolved in distilled water and stirred magnetically for 30 min to achieve a protein concentration of 10% (w/v), it was placed overnight at 4° C. for sufficient hydration.
(2) The protein solution was heated in a 90° C. water bath for 30 min, and then quickly transferred to an ice-water bath to obtain heat induced gel.

### 2. Determination Method for Gel Strength

The gel sample was cut into an equal height cylinder with a diameter of 12 mm, and determined by a TA50 spherical probe at 30% deformation, 5.0 g of a trigger point load and a test speed of 0.5 mm/s. The gel strength was equal to the product of a breaking force (g) and a breaking distance (cm).

### 3. Result

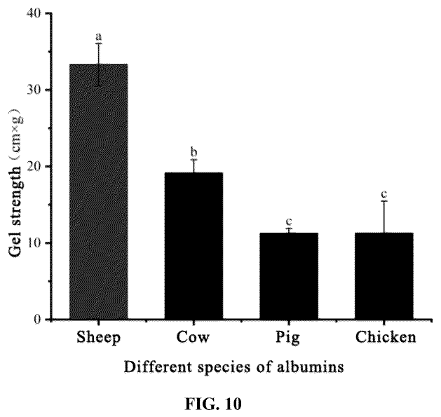

The results were shown in FIG. 10, compared with other species, the sheep serum albumin had the apparent significance in terms of the gel strength, and had the better gel strength.

The above details the preferred embodiments of the present invention. However, the invention is not limited to the specific details described herein. Within the scope of the technical concept, various simple modifications can be made to the technical solutions, all of which fall within the protection scope of the invention.

Additionally, it should be noted that specific technical features described in the embodiments may be combined in any suitable manner without contradiction. To avoid unnecessary repetition, the invention will not separately explain all possible combinations.

What is claimed is:

1. A method for rapidly separating a sheep serum albumin based on an aqueous two-phase system, comprising the following steps:
(1) adding the aqueous two-phase system to a sheep plasma, stilly placing after stirring a mixture obtained, centrifuging, and collecting upper phase solution, wherein the aqueous two-phase system consists of ethanol, sodium dihydrogen phosphate, and water, or the aqueous two-phase system consists of ethanol, sodium dihydrogen phosphate, water, and sodium chloride;
(2) removing the ethanol from the upper phase solution;
wherein a mass percentage of the sheep plasma in the mixture is 1-5%, a mass percentage of the ethanol in the mixture is 25-35%, a mass percentage of the sodium dihydrogen phosphate in the mixture is 16-24%, and a mass percentage of the sodium chloride in the mixture is 1-5%.

2. The method as claimed in claim 1, wherein the mass percentage of the sheep plasma in the mixture is 3%, the mass percentage of the ethanol in the mixture is 30%, the mass percentage of the sodium dihydrogen phosphate in the mixture is 20%.

3. The method as claimed in claim 1, wherein the mass percentage of the sheep plasma in the mixture is 2-4%, the mass percentage of the ethanol in the mixture is 25-30%, the mass percentage of the sodium dihydrogen phosphate in the mixture is 16-20%, and the mass percentage of the sodium chloride in the mixture is 1-5%.

4. The method as claimed in claim 3, wherein the mass percentage of the sheep plasma in the mixture is 4%, the mass percentage of the ethanol in the mixture is 28%, the mass percentage of the sodium dihydrogen phosphate in the mixture is 18%, and the mass percentage of the sodium chloride in the mixture is 3%.

5. The method as claimed in claim 1, wherein a standing time is 10-60 min.

6. The method as claimed in claim 1, wherein centrifugation conditions are: a centrifugation temperature is 1-10° C., a centrifugation speed is 2500-4500 rpm, and a centrifugation time is 5-25 min.

7. The method as claimed in claim 1, wherein the step (2) further comprises a freeze-drying operation.

8. The method as claimed in claim 7, wherein conditions for the freeze-drying operation are: a freeze-drying temperature is-90-70° C., and a freeze-drying time is 36-60 h.
